(19) United States
(12) Patent Application Publication
(10) Pub. No.: US 2014/0081157 A1
(43) Pub. Date: Mar. 20, 2014

(54) Apparatus and Method for determining a volume amount of a physiological volume

(71) Applicant: Stephan Joeken, Schopfheim (DE)

(72) Inventor: Stephan Joeken, Schopfheim (DE)

(21) Appl. No.: 13/520,311

(22) PCT Filed: Dec. 30, 2009
(86) PCT No.: PCT/EP09/68016

§ 371 Date: Dec. 3, 2013

(51) Int. Cl. A61B 5/0295; A61B 5/01; A61B 5/00; A61B 5/0205

(52) U.S. Cl. CPC A61B 5/0295; A61B 5/02055; A61B 5/01; A61B 5/7225
USPC 600/484; 600/507; 600/483

(57) ABSTRACT

An apparatus and a method for determining the volume amount of a physiological volume (EVLW) from the system response to two successive system disturbances, taking into account that an intrinsic physical property of the physiological volume is influenced by the first (or previous, respectively) system disturbance, is described. By introducing a cold bolus to the central venous blood stream, a flowed-by volume, such as extravascular lung water, is cooled down. The driving temperature gradient for heat transfer is reduced when a second cold bolus is introduced. From the difference of the system response to the first bolus injection and the second bolus injection EVLW can be determined.

The present invention relates to an apparatus and a method for determining at least one volume amount of a respective physiological volume flowed through and/or flowed by in a through-flow region by a blood stream, such as Extravascular Lung Water (EVLW), Intrathoracic Blood Volume (ITBV) and Global Enddiastolic Volume (GEDV). Such volume amount are parameters enabling the physician in charge to judge the present condition of the patient and to take appropriate counter measures, if the condition should worsen. For example, GEDV is used to assess the filling state of a patient, and EVLW is an important parameter for observing the development of a pulmonary oedema. To improve readability, the term “physiological volume” is also used for the volume amount of a physiological volume, i.e. “physiological volume” means both the actual physical entity, such as the extravascular water in the lungs and the blood in the thorax, and the value of the determined amount thereof.

Transpulmonary thermodilution techniques constitute the current state of the art for determining ITBV, GEDV and EVLW or, more precisely, an extravascular thermovolume representing an approximation of EVLW (which shall be considered equivalent herein-after). Patient monitoring apparatus implementing such techniques are commonly used in modern day hospitals for monitoring the condition of the circulatory system of critically ill patients. As is well known to the person skilled in the art, patient monitoring apparatus may combine transpulmonary thermodilution techniques with pulse contour analysis and/or other measurement approaches.

Transpulmonary thermodilution techniques and patient monitoring apparatus implementing such techniques for determining one or more of the above parameters are described inter alii in U.S. Pat. No. 5,526,817.

Generally, patient monitoring apparatus employing thermodilution techniques comprise control means for providing data characterising a temperature change imposed on travelling blood volume elements of the blood stream at a first location upstream of the physiological volume of interest, sensor means for measuring the temperature in said blood stream at a second location downstream of the physiological volume of interest, and evaluation means with an input channel for reading in measurement readings from the sensor means and storing means for storing said measurement readings over time. The evaluation means calculate the volume amount of the physiological volume of interest from the data characterising the imposed change and the measurement readings.

To monitor changes of the volume amount over time, the measurement can be repeated. Further, the measurement may be repeated imposing a different temperature change in order to improve accuracy of the measurements, as described in U.S. Pat. No. 6,394,961 and U.S. Pat. No. 6,537,230.

While usually injection of a cold bolus is used for imposing a temperature change on the travelling blood volume elements; U.S. Pat. No. 6,736,782 discloses use of catheter assemblies that can emit heat pulses to the blood stream.

As an alternative to thermodilution techniques or in combination therewith, dilution techniques may be employed using injection of other indicators such as dye or salt solutions in order to impose a change of an intrinsic physical property other than temperature (e.g. conductivity or optical properties) on travelling blood volume elements.

Conventional dilution techniques are thus based on, analysing the system response to a disturbance introduced by injecting a bolus or emitting a heat pulse. In the conventional dilution algorithms known from the prior art, a linear system response is assumed, i.e. a linear relation between the volume amount of the physiological volume of interest and the measured intrinsic physical property is employed.

While conventional dilution techniques generally perform well determining the volume amount of a physiological volume at an isolated point in time, they have shown to become inaccurate if measurements are repeated within a short time span. In particular, they appear not to be well-suited for quasi-continuously monitoring the respective volume amount.

It is therefore an object of the present invention, to decrease the time span within which a dilution measurement can be repeated with sufficient accuracy. Further, it is an object of the present invention to generally increase accuracy and dependability of volume amount of a respective physiological volume flowed through and/or flowed by in a through-flow region by a blood stream, such as Extravascular Lung Water (EVLW), Intrathoracic Blood Volume (ITBV) and Global. Enddiastolic Volume (GEDV) determination of physiological volumes, such as Extravascular Lung Water (EVLW), Intrathoracic Blood Volume (ITBV) and Global Enddiastolic Volume (GEDV).

To accomplish the above object, according to one aspect of the present invention, an apparatus for determining at least one volume amount of a respective physiological volume flowed through and/or flowed by in a through-flow region by a blood stream is provided, comprising

(a) control means for providing data characterising a first change of an intrinsic physical property of first travelling blood volume elements of the blood stream at a first location upstream of the through-flow region at a first point in time and data characterising a second change of the intrinsic physical property of second travelling blood volume elements of the blood stream at the first location at a second point in time later than said first point in time,
(b) sensor means for measuring the intrinsic physical property in the blood stream at a second location downstream of the through-flow region; and
(c) evaluation means comprising an input channel for reading in measurement readings from the sensor means and storing means for storing the measurement readings over time.

The evaluation means are adapted to calculate from the data characterising the first change, the data characterising the second change and the measurement readings the at least one volume amount, wherein the evaluation means are adapted to employ a non-linear relation between the at least one volume amount and the course of the intrinsic physical property at the second location over time as represented by the measurement readings. The non-linear relation models.
a change of an intrinsic physical property of the physiological volume due to heat and/or mass exchange occurring between the physiological volume and the first travelling blood volume elements in the through-flow region and
a resulting difference of heat and/or mass transfer occurring between the physiological volume and the second travelling blood volume elements in said through-flow region vis-à-vis heat and/or mass exchange occurring between the physiological volume and the first travelling blood volume elements in the through-flow region.

In other words, the present invention provides for determining the volume amount of a physiological volume from the system response to two (or more) successive system disturbances, taking into account that an intrinsic physical property of the physiological volume is influenced by the first (or previous, respectively) system disturbance. For example, by introducing a cold bolus to the central venous blood stream, a flowed-by volume, such as extravascular lung water, is cooled down. Therefore, the driving temperature gradient for heat transfer is reduced when a second cold bolus is introduced. From the difference of the system response to the first bolus injection and the second bolus injection EVLW can be determined.

According to a preferred embodiment, the control means include detection means for detecting a timing and a quantity of said first change and a timing and a quantity of said second change. E.g., the detection means may include a temperature sensor for determining a bolus temperature and a pressure switch or the like for determining the timing of a respective bolus injection.

According to another advantageous embodiment, the apparatus comprises imposing means for imposing the first change and the second change, such as injection means, heating means and/or cooling means, and the control means are adapted to actively control the imposing means.

According to another aspect of the present invention, the above object is accomplished by providing an evaluation method for determining at least one physiological volume flowed through and/or flowed by in a through-flow region by a blood stream. The method comprises the steps of

(i) providing data characterising a change of a physical variable of travelling blood volume elements of the blood stream at a location upstream of the through-flow region,
(ii) providing data characterising a second change of the intrinsic physical property of second travelling blood volume elements of the blood stream at the first location at a second point in time later than the first point in time,
(iii) reading in measurement readings indicative of the physical variable in the blood stream downstream of the through-flow region, and
(iv) storing the measurement readings over time.

The method further includes steps of calculating from the data characterising the first change, the data characterising the second change and the measurement readings the at least one volume amount. Therein, a non-linear relation between the at least one volume amount and the course of the intrinsic physical property at said second location over time as represented by said measurement readings is employed. The non-linear relation models
a change of an intrinsic physical property of said physiological volume due to heat and/or mass exchange occurring between the physiological volume and the first travelling blood volume elements in the through-flow region and
a resulting difference of heat and/or mass transfer occurring between the physiological volume and the second travelling blood volume elements in the through-flow region vis-à-vis heat and/or mass exchange occurring between the physiological volume and the first travelling blood volume elements in the through-flow region.

In this context, providing data characterising an imposed change can either include reading in respective data resulting from detection of a timing and a quantity of the respective change, or reading out respective data for actively controlling imposing means to impose the respective change, or both.

In advantageous embodiments of the present invention, the at least one volume amount includes at least one of Extravascular Lung Water (EVLW), Intrathoracic Blood Volume ITBV and Global Enddiastolic Volume (GEDV).

While said first change usually will be a change of an intrinsic physical property imposed on the first travelling blood volume elements of the blood stream at the first location and said second usually will be a change of an intrinsic physical property imposed on the second travelling blood volume elements of the blood stream at the first location, the invention may also be advantageously carried out on the basis of arbitrary changes of the respective physical property occurring in the blood stream.

Implementation of the non-linear relation can advantageously be based on the following:

### If

Y(t) is the intrinsic physiological property measured at the second location downstream of the through-flow region (system response),
f (t−τ₁) is a function describing the (hypothetical) response to the first imposed change of the intrinsic physical property at the first location at a time τ₁ (such as a Dirac function),
f (t−τ₂) is a function describing the hypothetical linear response to the second imposed change of the intrinsic physical property at the first location at a time τ₂ (such as a Dirac function),
g (t−(τ₂τ₁)) the difference between the actual (non-linear) system response and a hypothetical (linear) system response that could be expected if the intrinsic physical property of the physiological volume did not change due to heat and/or mass exchange occurring between the physiological volume and the first travelling blood volume elements in the through-flow region,
then the following applies:

Y(t)=f(t−τ₁)+f(t−τ₂)+g(t−(τ₂−τ₁)).

The above is a particular cascade-model of the more general embodiment wherein the system response is advantageously described by the Volterra-Series:

Y(t)=Y₀+∫f(Σ)X(t−τ)dτ+∫∫g(τ₁,τ₂)X(t−τ₁)X(t−τ₂)dτ₁dτ₂+

wherein the (imposed) changes of the intrinsic physical property at the first location is X(t)=δ(t−τ₁)+δ(t−τ₂), with Dirac function δ and constant offset Y₀ (e.g. baseline body temperature T₀).

If the changes of the intrinsic physical property X(t) at the first location represented by Gaussian white noise the above integral series becomes the Wiener-Series. However, advantageously for any intrinsic physical property X, the non-linear relation includes a relation of the form

∫∫g(τ₁,τ₂)X(t−τ₁)X(t−τ₂)dτ₁dτ₂.

Advantageously, assuming that a Wiener-Series is applicable, g(τ₁,τ₂) can be derived employing a cross-correlation of the intrinsic physical property X(t) at the first location and the system response Y(t) at the second location. Therein, if <argument> indicates the mean of the argument and σ² the variance, then for the integral kernels applies

Y₀=<Y(t)>

f(τ)=1/σ²<Y(t)X(t−τ)>

and

g(τ₁,τ₂)=1/(σ²)²<Y(t)X(t−τ₁)X(t−τ₂)>−1/(2σ²)Y₀δ(σ₁−τ₂).

In a particularly preferred embodiment, g(τ₁,τ₂) is represented by a system of orthogonal functions, and hence determination of g(τ₁,τ₂) is straight forward, see e.g. Korenberg, M J et al. (1988), Ann. Biomed. Eng. 16, 201-214 and Korenberg, M J (1989), Biol. Cybern. 60, 267-276.

In a particularly preferred embodiment of the present invention, the physical variable is temperature. In this case, the sensor means will comprise a temperature sensor.

As the skilled person will understand, the invention is not limited to only two imposed changes to a physical property of blood volume elements. Instead, the invention can be carried out imposing multiple changes to respective blood volume elements. E.g., using a catheter adapted to repeatedly emit (positive or negative) heat pulses, employing a Peltier element, a heating coil or the like, will allow to implement the present invention for quasi-continuous physiological volume determination.

Therefore, the invention more generally also provides for an apparatus for determining at least one volume amount of a respective physiological volume flowed through and/or flowed by in a through-flow region by a blood stream, comprising

(a) control means for providing data characterising a plurality of changes of an intrinsic physical property of respective travelling blood volume elements of the blood stream at a first location upstream of the through-flow region at respective points in time,
(b) sensor means for measuring the intrinsic physical property in the blood stream at a second location downstream of the through-flow region,
(c) evaluation means comprising an input channel for reading in measurement readings from the sensor means and storing means for storing the measurement readings overtime,
wherein the evaluation means are adapted to calculate from the data characterising the plurality of changes and the measurement readings the at least one volume amount employing a non-linear relation between the at least one volume amount and the course of the intrinsic physical property at the second location over time as represented by the measurement readings.

Therein; the non-linear relation models
a change of an intrinsic physical property of the physiological volume due to heat and/or mass exchange occurring between the physiological volume and previous travelling blood volume elements in the through-flow region and
a resulting difference of heat and/or mass transfer occurring between the physiological volume and subsequent travelling blood volume elements in the through-flow region vis-à-vis heat and/or mass exchange occurring between the physiological volume and the previous travelling blood volume elements in the through-flow region.

Likewise, the invention more generally also provides for an evaluation method for determining at least one volume amount of a respective physiological volume flowed through and/or flowed by in a through-flow region by a blood stream, comprising steps of

(i) providing data characterising successive changes of an intrinsic physical property of respective blood volume elements of the blood stream at a first location upstream of the through-flow region,
(ii) reading in measurement readings indicative of the physical variable in the blood stream downstream of the through-flow region,
(iv) storing the measurement readings over time,
wherein the method further includes steps of calculating from the data characterising the plurality of changes and the measurement readings the at least one volume amount employing a non-linear relation between the at least one volume amount and the course of the intrinsic physical property at the second location over time as represented by the measurement readings.

Therein, the non-linear relation models
a change of an intrinsic physical property of the physiological volume due to heat and/or mass exchange occurring between the physiological volume and previous travelling blood volume elements in the through-flow region and
a resulting difference of heat and/or mass transfer occurring between the physiological volume and subsequent travelling blood volume elements in the through-flow region vis-à-vis heat and/or mass exchange occurring between the physiological volume and the previous travelling blood volume elements in the through-flow region.

Again, while said changes of an intrinsic physical property of respective blood volume elements of the blood stream at the first location usually will be a changes of the intrinsic physical property imposed on the respective travelling blood volume elements, the invention may also be advantageously carried out on the basis of arbitrary changes of the respective physical property occurring in the blood stream.

Generally, any of the embodiments described or options mentioned herein may be particularly advantageous depending on the actual conditions of application. Further, features of one embodiment may be combined with features of another embodiment as well as features known per se from the prior art as far as technically possible and unless indicated otherwise.

The invention will now be described in more detail. The accompanying drawings, which are schematic illustrations, serve for a better understanding of the features of the present invention. Therein

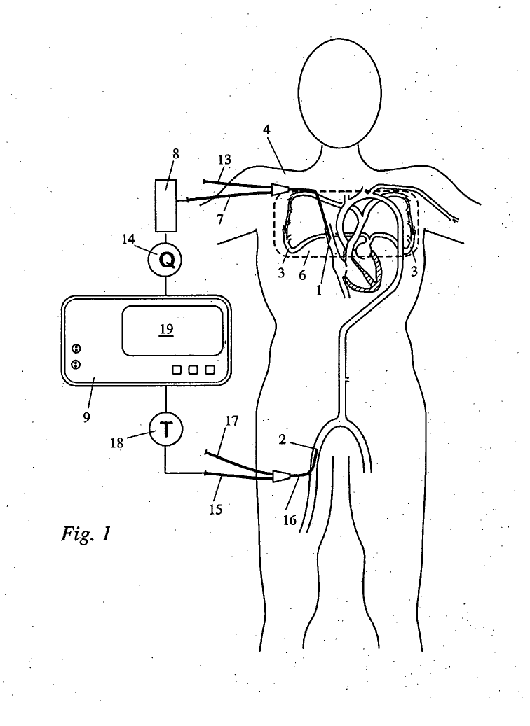

FIG. 1 shows an exemplary setup of an advantageous embodiment of the present invention,

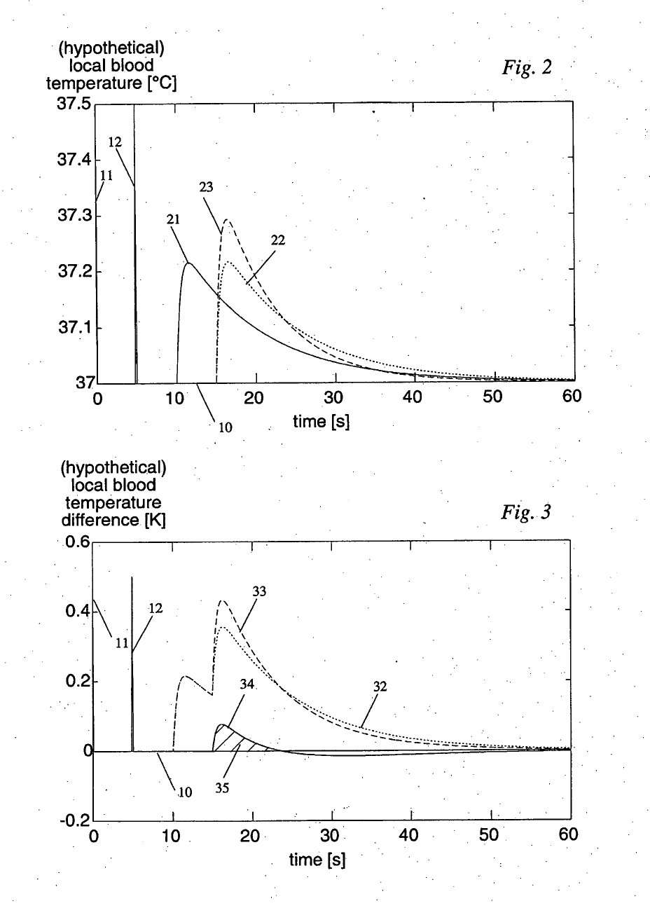

FIG. 2 is a diagram of the local blood temperature at a first (upstream) location, where two temperature changes are imposed and hypothetically resulting respective blood temperatures at a second (downstream) location.

FIG. 3 is a diagram of the local blood temperature difference vis-à-vis the base blood temperature at a first (upstream) location, where two temperature changes are imposed, and both an actual and a hypothetical resulting blood temperature at a second (downstream) location.

In FIG. 1, a preferred embodiment of the present invention is depicted. Therein, the physiological volume of interest is the extravascular lung water 6 of the patient 4, i.e. the volume amount EVLW thereof is to be determined. The patient's 4 blood flow is in thermal contact with the extravascular lung water 6 in the pulmonary circulation 3. The pulmonary circulation can thus be considered the through flow region 3 in the above sense.

A central venous, catheter assembly 7 is, provided for imposing local blood temperature changes on the patient's 4 blood stream in the vena cava superior 1, i.e. at a first location 1 upstream of the through-flow region 3. The central venous catheter assembly 7 is equipped with imposing means 8 for imposing the local blood temperature changes. These imposing means 8 may comprise an injector for cold (or heated) boli as well as a heating or cooling element (such as heating coil or a peltier element). If bolus injection means are used, it is advantageous to provide a temperature sensor for detecting the temperature of the injected bolus. The timing of bolus injections may be detected by a pressure switch or the like. If an automated injector is implemented, than the timing of the control signals initiating and/or ending injection is recorded by the patient monitoring apparatus 9, which also comprises evaluation means. If electrical heating or cooling means are implemented, the heat pulse emission (or cooling) can be characterized by the respective electrical power supplied and the timing of supplying said power.

The patient monitoring apparatus 9 comprises a channel 14 for controlling the imposing means 8. Thus, in the present embodiment, the patient monitoring apparatus 9 also includes control means.

The central venous catheter assembly 7 may comprise additional ports and lumina 13 for pressure measurements, injection of medication, blood sample withdrawal, optical probes or the like.

An arterial catheter assembly 15 is provided for measuring local blood temperature in the femoral artery 2, i.e. at a second location 2 downstream of the through flow region 3 over time. For this purpose, the arterial catheter assembly 15 comprises sensor means, such as a thermistor 16. The arterial catheter assembly 15 may comprise additional ports and lumina 17 for pressure measurements, blood sample withdrawal, optical probes or the like.

The patient monitoring apparatus 9 comprises an input channel 18 for reading in measurement readings from the thermistor 16.

The patient monitoring apparatus 9 further comprises a microcomputer which is adapted to calculate EVLW employing a non-linear relation between EVLW and the course of the blood temperature at the second location 2 over time as represented by the measurement readings. The resulting EVLW can be displayed on the display 19.

The non-linear relation models a change of temperature of extra vascular lung water 6 due to heat exchange occurring between the extra vascular lung water 6 and previous travelling blood volume elements in the through-flow region 3 and a resulting difference of heat transfer occurring between extra vascular lung water 6 and subsequent travelling blood volume elements in the through-flow region 3 vis-à-vis heat exchange occurring between the extra vascular lung water 6 and the previous travelling blood volume elements in the through-flow region 3. An example of such a non-linear relation is explained in connection with FIGS. 2 and 3.

FIGS. 2 and 3 illustrate the basic principle of the present invention for an example wherein the invention is carried out imposing two successive heat pulses.

FIG. 2 is a diagram of the local blood temperature over time, both at a first location (e.g. the vena cava superior) 1 upstream of the through-flow region 3, where the blood stream of the patient 4 is in thermal contact with the extravascular lung water, and a second location (e.g. the femoral artery) 2 downstream of the through flow region 3 over time. The abszissa thus represents time and the ordiante blood temperature.

Therein, the blood temperatures at the second location 2 are merely hypothetical and intended to serve illustrative purposes only, as explained below. A first temperature peak 11 (not visible as it coincides with the ordinate) and a second temperature peak 12 are imposed on the blood stream at the first location 1 within a short period of time (5 seconds in this example). The peaks shown correspond to respective heat pulses, but the principles explained herein also apply for local cooling (e.g. by cold bolus injection or a cooling element such as peltier element), in which case the peaks 11, 12 and curves 21, 22, 23, would be mirrored downward at the blood temperature baseline 10.

The continuous line 21 shows a hypothetical system response to the first peak 11 measured at the second location 2, if the second peak 12 did not occur. In other words, continuous line 21 indicates the portion of the overall system response that results from the heat emitted during the heat pulse corresponding to the first peak 11. The dotted line 22 shows a hypothetical system response to the second peak 12 measured at the second location 2, if the first peak 11 did not occur. In other words, dotted line 22 indicates the portion of the overall system response that results from the heat emitted during the heat pulse corresponding to the second peak 12. Therein, it has been disregarded that the extravascular lung water has been heated up as a result from the heat pulse corresponding to the first peak 11, because heat is transferred to the extravascular lung water, while the blood volume elements heated by the heat pulse are travelling through the through flow region 3.

Actually, due to the increased temperature of the extravascular lung water, the driving temperature gradient for heat transfer is smaller, when the blood volume elements heated by the heat pulse corresponding to the second peak 12 are travelling through the through flow region 3, than the driving temperature gradient was, when the blood volume elements heated by the heat pulse corresponding to the first peak 12 were travelling through the through flow region 3. As a result, the portion of the overall system response that results from the heat emitted during the heat pulse corresponding to the second peak 12 rather corresponds to the curve indicated by broken line 23. In particular, the second hypothetical response indicated by broken line 23 is shifted towards the left (i.e. towards shorter times).

However, as cardiac output is assumed to remain unchanged, the respective areas under the curve indicated by the dotted line 22 and under the curve indicated by the broken line 23 are equal.

In FIG. 3, dotted line 32 represents a hypothetical system response assuming a linear system behaviour, i.e. dotted line 32 represents the summation of hypothetical portions indicated by continuos line 21 and dotted line 22 of FIG. 2. Therein, the scale of the ordinate has been changed by relating to a temperature difference vis-à-vis the blood temperature baseline 10 rather than relating to blood temperature.

Broken line 33 represents an actual system response assuming a non-linear system behaviour, i.e. broken line 33 represents the summation of hypothetical portions indicated by continuos line 21 and broken line 23 of FIG. 2.

The difference between the actual (non-linear) system response indicated by broken line 33 and the hypothetical (linear) system response indicated by dotted line 32 is an indication of the volume amount EVLW of extra vascular lung water 6. This difference is indicated by continuous line 34 in FIG. 3.

In particular, the hatched area 35 under continuous line 34 (i.e. the integral over the positive stretch of the curve) is proportional to the volume amount EVLW of extra vascular lung water 6.

This difference between the actual (non-linear) system response indicated by broken line 33 and the hypothetical (linear) system response indicated by dotted line 32, as represented by continuous line 34 in FIG. 3, can be described by series of integrals (Wiener series or Volterra series) and can be determined utilising orthogonalization methods.

### Be

T(t) the temperature T measured at the second location 2 downstream of the through flow region 3 at a time t,
f (t−τ₁) the (hypothetical) response to a first bolus injected or a first heat pulse emitted at a time τ₁,
f (t−τ₂) the hypothetical linear response to a second bolus injected or a second heat pulse emitted at a time τ₂,
g (t−(τ₂−τ₁)) the difference between the actual (non-linear) system response and the hypothetical (linear) system response, as represented by continuous line 34 in FIG. 3,
then the following applies for the above described example:

T(t)=f(t−τ₁)+f(t−τ₂)+g(t−(τ₂−τ₁)).

1. An apparatus for determining at least one volume amount of a respective physiological volume flowed through and/or flowed by in a through-flow region by a blood stream, comprising
a controller for providing data characterising at least two changes of an intrinsic physical property of respective travelling blood volume elements of said blood stream at a first location upstream of said through-flow region at respective points in time,
a sensor for measuring said intrinsic physical property in said blood stream at a second location downstream of said through-flow region,
an evaluator comprising an input channel for reading in measurement readings from the sensor and storage for storing said measurement readings over time,
wherein the evaluator is adapted to calculate from said data characterising said changes and said measurement readings said at least one volume amount employing a non-linear relation between said at least one volume amount and the course of said intrinsic physical property at said second location over time as represented by said measurement readings,
said non-linear relation models a change of an intrinsic physical property of said physiological volume due to heat and/or mass exchange occurring between said physiological volume and previous travelling blood volume elements in said through-flow region, and
a resulting difference of heat and/or mass transfer occurring between said physiological volume and subsequent travelling blood volume elements in said through-flow region vis-à-vis heat and/or mass exchange occurring between said physiological volume and said previous travelling blood volume elements in said through-flow region.

2. The apparatus according to claim 1, wherein said at least one volume amount includes at least one of Extravascular Lung Water (EVLW), Intrathoracic Blood Volume ITBV and Global Enddiastolic Volume (GEDV).

3. The apparatus according to claim 1, wherein said controller includes a detector for detecting a respective timing and a respective quantity of each of said changes of said intrinsic physical property of said respective travelling blood volume elements.

4. The apparatus according to claim 1, wherein said apparatus further comprises imposing means for imposing said changes of said intrinsic physical property of said respective travelling blood volume elements, wherein said controller is adapted to actively control said imposing means.

5. The apparatus according to claim 4, wherein said imposing means include injection means.

6. The apparatus according to claim 4, wherein said imposing means include heating means and/or cooling means.

7. The apparatus according to claim 1, wherein said non-linear relation includes a relation of the form
∫∫g(τ₁,τ₂)X(t−τ₁)X(t−τ₂)dτ₁dτ₂;
wherein X is said intrinsic physical property at said first location and t is time, g(τ₁,τ₂) is a function term describing said non-linear relation.

8. The apparatus according to claim 7, wherein g (τ₁, τ₂) is derived employing a cross-correlation of the intrinsic physical property at said first location and the system response measured at said second location.

9. The apparatus according to claim 7, wherein g(τ₁,τ₂) is represented by a system of orthogonal functions.

10. The apparatus according to claim 9, wherein g(τ₁,τ₂) is derived employing said orthogonal functions.

11. The apparatus according to claim 1, wherein said controller is adapted to actively control said imposing means.

12. The apparatus according to claim 1, wherein said intrinsic physical property is temperature and said sensor comprises a temperature sensor.

13. The apparatus according to claim 1, wherein said apparatus is adapted for ongoing determination of said at least one volume amount on the basis of a plurality of changes of said intrinsic physical property of respective travelling blood volume elements.

14. An evaluation method for determining at least one volume amount of a respective physiological volume flowed through and/or flowed by in a through-flow region by a blood stream, the method comprising
providing data characterising at least two successive changes of an intrinsic physical property of respective travelling blood volume elements of said blood stream at a first location upstream of said through-flow region,
reading in measurement readings indicative of said physical variable in said blood stream downstream of said through-flow region,
storing said measurement readings over time,
wherein said method further includes steps of calculating from said data characterising said changes and said measurement readings said at least one volume amount employing a non-linear relation between said at least one volume amount and the course of said intrinsic physical property at said second location over time as represented by said measurement readings,
said non-linear relation models a change of an intrinsic physical property of said physiological volume due to heat and/or mass exchange occurring between said physiological volume and previous travelling blood volume elements in said through-flow region, and
a resulting difference of heat and/or mass transfer occurring between said physiological volume and subsequent travelling blood volume elements in said through-flow region vis-à-vis heat and/or mass exchange occurring between said physiological volume and the previous travelling blood volume elements in said through-flow region.

15. The method according to claim 14, wherein said at least one volume amount includes at least one of Extravascular Lung Water (EVLW), Intrathoracic Blood Volume ITBV and Global Enddiastolic Volume (GEDV).

16. The method according to claim 14, wherein said non-linear relation includes a relation of the form
∫∫g(τ₁,τ₂)X(t−τ₁)X(t−τ₂)dτ₁dτ₂;
wherein X is said intrinsic physical property at said first location and t is time, g(τ₁,τ₂) is a function term describing said non-linear relation.

17. The method according to claim 16, wherein g(τ₁,τ₂) is derived employing a cross-correlation of the intrinsic physical property at said first location and the system response measured at said second location.

18. The method according to claim 17, wherein g(τ₁,τ₂) is represented by a system of orthogonal functions.

19. The method according to claim 18, wherein g(τ₁,τ₂) is determined by means of said orthogonal functions.

20. The method according to claim 14, wherein said intrinsic physical property is temperature.

21. The method according to claim 14, wherein said at least one volume amount is determined repeatedly on the basis of a plurality of changes of said intrinsic physical property of respective travelling blood volume elements.

22. The method according to claim 14, wherein said method further includes imposing said changes of said intrinsic physical property on respective travelling blood volume elements.

23. The method according to claim 22, wherein said method further includes applying disposable imposing means to a patient for imposing said changes.

24. The method according to claim 14, wherein said method further includes measuring said physical variable in said blood stream downstream of said through-flow region.

25. The method according to claim 24, wherein said method further includes applying a disposable sensor to a patient for measuring said physical variable in said blood stream downstream of said through-flow region.
